(19) United States
(12) Patent Application Publication
(10) Pub. No.: US 2006/0079706 A1
(43) Pub. Date: Apr. 13, 2006

(54) Bicalutamide polymorphs

(75) Inventors: Reddy Bandi Parthasaradhi, Andhrapradesh (IN); Reddy Kura Rathnakar, Andhrapradesh (IN); Reddy Rapolu Raji, Andhrapradesh (IN); Reddy Attunuri Narasa, Andhrapradesh (IN); Reddy Bolla Narasa, Andhrapradesh (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: Hetero Drugs Limited, Andhrapradesh (IN)

(21) Appl. No.: 11/289,792

(22) Filed: Nov. 30, 2005

Related U.S. Application Data
(62) Division of application No. 10/450,103, filed on Jun. 10, 2003, now abandoned.

(51) Int. Cl. A61K 31/277

(52) U.S. Cl. 558/413; 514/521

(57) ABSTRACT

The invention provides crystalline form of bicalutamide and amorphous bicalutamide. The invention also provides methods for their preparation and pharmaceutical compositions containing the new forms of bicalutamide.

This application is a division application of application Ser. No. 10/450,103, filed on Jun. 10, 2003, entitled BICALUTAMIDE POLYMORPHS, and whose entire disclosure is incorporated by reference herein.

### FILELD OF THE INVENTION

The present invention provides a bicalutamide crystalline form and amorphous bicalutamide. The present invention also provides methods of preparing these forms.

### BACKGROUND OF THE INVENTION

Bicalutamide which is known by the chemical name N-[4-cyano-3-(trifluoromethyl)phenyl]-3-[(4-fluorophenyl)sulfonyl]-2-hydroxy-2-methylpropanamide is used for treatment of prostate cancer which is described in U.S. Pat. No. 4,636,505. Various methods of synthesis of bicalutamide are disclosed in U.S. Pat. No. 6,479,692, WO 01/00608, US patent application No.2002/0086902.

In all the prior art documents bicalutamide is crystallized from ethyl acetate/petroleum ether. Bicalutamide crystallized from ethyl acetate/petroleum ether does not produce well defined, stable polymorphic form. A well-defined crystalline form of bicalutamide is synthesized and characterized. According to the present invention, the new crystalline form is found to be obtainable in pure form and stable and consistently reproducible.

Prior art does not disclose amorphous form of bicalutamide and also, processes described in the prior art does not produce amorphous form of bicalutamide. The amorphous forms in a number of drugs exhibit different dissolution characteristics and in some cases different bioavailability patterns compared to the crystalline form. For some therapeutic indications one bioavailability pattern may be favored over another. According to the present invention amorphous form of bicalutamide with good dissolution characteristics is synthesized.

Thus, the object of present invention is to provide a stable, pure, consistently obtainable crystalline form of bicalutamide methods for preparing bicalutamide crystalline form and pharmaceutical formulations containing bicalutamide crystalline form.

Another object of the present invention is to provide a stable amorphous bicalutamide and converting the bicalutamide crystalline form into amorphous form of bicalutamide.

### SUMMARY OF THE INVENTION

The present invention provides N-[4-cyano-3-(trifluoromethyl)phenyl]-3-[(4-fluoro-phenyl)sulfonyl]-2-hydroxy-2-methylpropanamide crystalline form (hereinafter referred to as bicalutamide crystalline form) having a typical x-ray diffraction pattern of FIG. 1. The significant reflections of the bicalutamide crystalline form are shown in Table 1 wherein d represents the interplanar spacing and I/I₁ represents the relative intensities expressed as a percentage of most intense reflection.

Another feature of the invention is to provide a method of preparing bicalutamide crystalline form which comprises:
i) dissolving bicalutamide obtained by a known method in a suitable solvent,
ii) maintaining the solution obtained in step (i) at 0-40° C. for about 5 to 36 hours, optionally seeded with bicalutamide crystalline form,
iii) filtering and drying the crystals formed to give bicalutamide crystalline form,
wherein suitable solvents include C₁-C₃ alcohol, C₁-C₆ ketone or mixture thereof. Preferred alcohols are ethanol, isopropyl alcohol and preferred ketone is acetone.

Another feature of the invention is to provide a pharmaceutical composition comprising the bicalutamide crystalline form.

Another feature of the invention is to provide amorphous form of bicalutamide (hereinafter referred to as amorphous bicalutamide) which is characterized by broad x- ray diffraction maxima at about 10.0 to 35.0 degrees 2θ. The typical x-ray diffractogram is shown in FIG. 2.

Another feature of the invention is to provide a process for preparation of amorphous bicalutamide which comprises:
i) heating bicalutamide to melt,
ii) cooling the mass to 25-35° C.,
iii) crushing the flakes formed in step (ii) to give amorphous bicalutamide
wherein bicalutamide used in step(l) is either bicalutamide obtained by a known method or bicalutamide crystalline form.

Another feature of the present invention is to provide an alternative method of preparing amorphous bicalutamide which comprises:
i) mixing bicalutamide and suitable solvent in a suitable proportion,
ii) slurring for about 1 to 5 hours,
iii) drying to give amorphous bicalutamide,
wherein suitable solvents include C₁-C₃ alcohol or C₁-C₆ ketone.

Bicalutamide used in step (i) is either bicalutamide obtained by a known method or bicalutamide crystalline form. Preferred alcohols are ethanol, isopropyl alcohol and preferred ketone is acetone.

The term “suitable proportion” implies that the weight/volume ratio of bicalutamide to the solvent is 1:2 to 1:8.

Another feature of the invention is to provide a pharmaceutical composition comprising the amorphous bicalutamide.

### BRIEF DESCRIPTION OF THE DRAWINGS

FIG. 1 is a powder x-ray diffractogram of bicalutamide crystalline form.

FIG. 2 is a powder x-ray diffractogram of amorphous bicalutamide. x-ray diffraction patterns were measured on a siemens D-5000 diffractometer with CuKr radiation.

### DETAILED DESCRIPTION OF THE INVENTION

Bicalutamide Crystalline Form

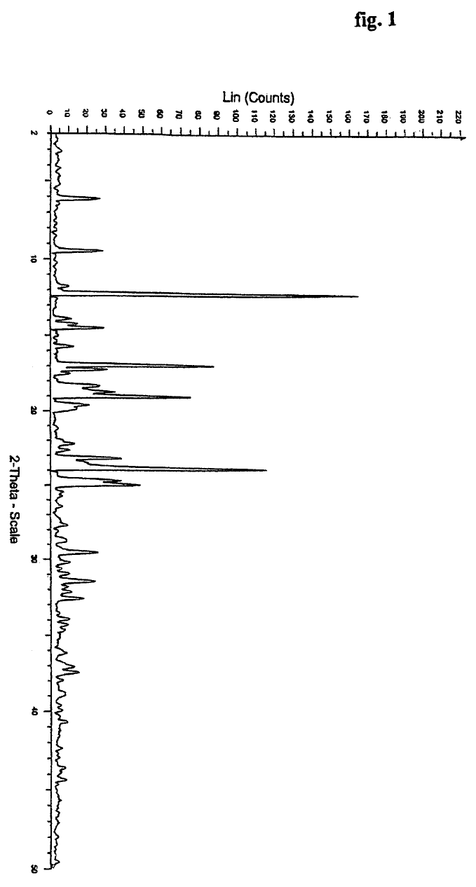

The present invention provides N-[4-cyano-3-(trifluoromethyl)phenyl]-3-[(4-fluorophenyl)sulfonyl]-2-hydroxy-2-methylpropanamide crystalline form (bicalutamide crystalline form) having a typical x-ray diffraction pattern of FIG. 1. The significant reflections of the bicalutamide crystalline form are shown in Table 1 wherein d represents the interplanar spacing and I/I₁ represents the relative intensities expressed as a percentage of most intense reflection.

Another feature of the invention is to provide a method of preparing crystalline form of bicalutamide.

Thus, bicalutamide obtained from a known method is dissolved in a suitable solvent. Suitable solvents include C₁-C₃ alcohol or C₁-C₆ ketones, preferred alcohols being ethanol, isopropyl alcohol and preferred ketones being acetone. The solution obtained is maintained at 0-40° C. for about 5 to 36 hours. Preferably, the solution is maintained at 20-35° C. for about 20-25 hours. During maintenance the solution may be seeded with bicalutamide crystalline form. The crystals formed are then filtered and dried to give bicalutamide crystalline form.

Amorphous Bicalutamide

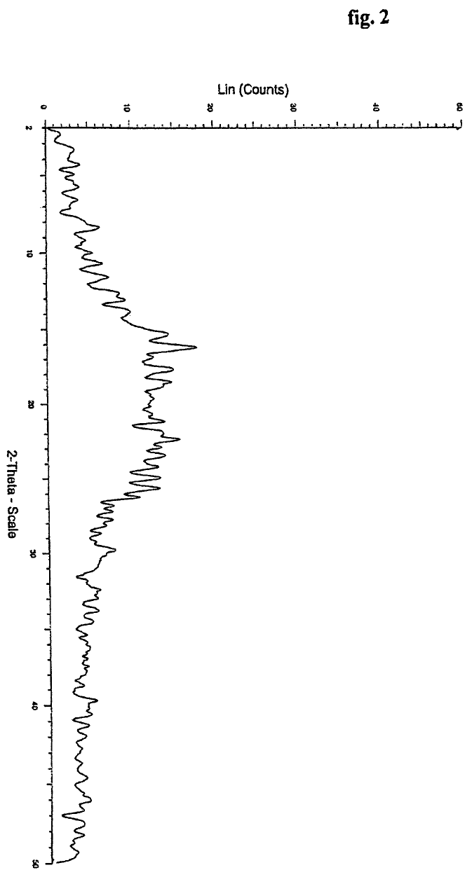

Another feature of the invention is to provide amorphous bicalutamide, which is characterized by broad x-ray diffraction maxima at about 10.0 to 35:0 degrees 2θ. The typical x-ray diffractogram is shown in FIG. 2.

Another feature of the invention is to provide a process for preparation of amorphous bicalutamide.

Thus, bicalutamide obtained by a known process or bicalutamide crystalline form is heated to about 195-200° C. to melt and then the mass is cooled gradually to 25-35° C. to form flakes. The flakes are crushed to give amorphous bicalutamide.

Another feature of the present invention is to provide an alternative method of preparing amorphous bicalutamide.

Thus, bicalutamide obtained by a known process or bicalutamide crystalline form is mixed with a suitable solvent in a suitable proportion. Suitable solvents include C₁-C₃ alcohol, C₁-C₆ ketones, and preferable alcohols being ethanol, isopropyl alcohol and preferable ketone being acetone. Suitable proportion implies that the weight/volume ratio of bicalutamide to the solvent is 1:2 to 1:8. The contents are slurried for about 1 to 5 hours and then dried to form amorphous bicalutamide. The drying can be of vacuum drying or spray drying.

Another feature of the invention is to provide a pharmaceutical composition comprising bicalutamide crystalline form.

Another feature of the invention is to provide a pharmaceutical composition comprising amorphous bicalutamide.

The compositions containing bicalutamide crystalline form or amorphous bicalutamide may be in a form suitable for oral dosage as a tablet, capsule, suspension, ointment, lotion. Any conventional technique may be used for the preparation of pharmaceutical formulation according to the invention. Examples of suitable diluents include lactose, micro crystalline cellulose, starch, mannitol. Examples of binders include polyvinyl pyrrolidone, hydroxy propyl cellulose, hydroxy propyl methylcellulose, methyl hydroxy propyl cellulose. Examples of suitable disintigrants include sodium starch glycollate, crospovidone, croscarmellose sodium. Examples of lubricants include magnesium stearate, zinc stearate, calcium stearate.

TABLE I

| d(A⁰) | Intensity(%) |
|---|---|
| 14.59071 | 16.4 |
| 9.40008 | 17.2 |
| 7.25084 | 100.0 |
| 6.13396 | 17.5 |
| 5.24717 | 53.0 |
| 5.15848 | 18.8 |
| 4.85606 | 16.2 |
| 4.74963 | 21.4 |
| 4.67733 | 45.6 |
| 4.53665 | 12.8 |
| 3.84215 | 23.3 |
| 3.73374 | 70.0 |
| 3.61162 | 23.3 |
| 3.57288 | 29.3 |
| 3.02588 | 15.5 |
| 2.84502 | 14.5 |
| 2.74755 | 10.9 |

The following nonlimiting examples illustrate the inventors preferred methods for preparing the compounds of the invention.

### EXAMPLES

### Example-1

m-Chloroperbenzoic acid (3 gm of 85% strength) was added in portion to a stirred solution of N-[4-cyano-3-(trifluoromethyl)phenyl]-3-[(4-fluorophenyl)thio]-2-hydroxy-2-methylpropanamide (2.7 gm) in methylene dichloride (450 ml). The reaction mixture is stirred at room temperature for 16 hours and then washed with saturated sodium sulfite solution (100 ml), aqueous sodium carbonate solution and brine and dried with Na₂SO₄. The solid obtained on removal of solvent was crystallized from ethyl acetate and petroleum ether (bp 60-80° C.) to give 2.5 gm of bicalutamide.

### Example-2

Bicalutamide (10 gm) obtained by the process described in example 1 was dissolved in acetone (50 ml) and the solution was stirred at 25-30° C. for 24 hours. The crystals formed were filtered and dried under vacuum to give 8.8 gm of bicalutamide crystalline form.

### Example-3

Crystalline form of bicalutamide (5 gm) by the process described in example 1, was heated to melt and the resulting transparent flake was crushed to give white powder of the amorphous bicalutamide in near quantitative yield.

### Example-4

Bicalutamide crystalline form (5 gm) obtained by the process described in example 2, was slurried in acetone (25 ml) for 2 hours and dried in vacuum to give white powder amorphous form of bicalutamide in near quantitative yield.

### Example-5

Amorphous bicalutamide (5 gm) obtained by the process described in example 1 was slurried in ethanol (30 ml) for 3 hours and spray dried to give white amorphous bicalutamide in near quantitative yield.

### Example-6

Example-2 was repeated by seeding the contents with bicalutamide crystalline form during stirring at 25 to 30° C. after 12 hours to give 9.2 gm of bicalutamide crystalline form.

We claim

1) Bicalutamide crystalline form characterized by a powder x-ray diffraction pattern having characteristic interplanar spacings shown in table 1.

2) Bicalutamide crystalline form characterized by an x-ray powder diffraction pattern of FIG. 1.

3) A method of preparing bicalutamide crystalline form which comprises:
i) dissolving bicalutamide obtained by known method in a suitable solvent,
ii) maintaining the solution obtained in step(i) at 0-40° C. for about 5 to 36 hours, optionally seeded with bicalutamide crystalline form,
iii) filtering and drying the crystals formed to give bicalutamide crystalline form,
wherein suitable solvents include C₁-C₃ alcohol, C₁-C₆ ketone.

4) A method according to claim 3 wherein suitable solvent is ethanol.

5) A method according to claim 3 wherein suitable solvent is acetone.

6) A method according to claim 3 wherein the solution is maintained at 25-30° C. for 20-25 hours in step (ii).

7) A method according to claim 3 wherein the solution is seeded with bicalutamide crystalline form.

8) Amorphous bicalutamide.

9) Amorphous bicalutamide characterized by a broad x-ray diffraction maxima at about 10.0 to 35.0 degree 2θ.

10) Amorphous bicalutamide having characteristic x-ray powder diffraction of FIG. 2.

11) A pharmaceutical composition comprising bicalutamide crystalline form and a pharmaceutically acceptable carrier.

12) A pharmaceutical composition comprising amorphous bicalutamide and a pharmaceutically acceptable carrier.
